(19) United States
(12) Patent Application Publication
(10) Pub. No.: US 2025/0107983 A1
(43) Pub. Date: Apr. 3, 2025

(54) Personal Care Compositions

(71) Applicant: Colgate-Palmolive Company, New York, NY (US)

(72) Inventors: Devon Sean COCUZZA, Flemington, NJ (US); Jin NAMKOONG, High Bridge, NJ (US); Qiang WU, Hillsborough, NJ (US)

(73) Assignee: Colgate-Palmolive Company, New York, NY (US)

(21) Appl. No.: 18/902,326

(22) Filed: Sep. 30, 2024

Related U.S. Application Data
(60) Provisional application No. 63/586,691, filed on Sep. 29, 2023.

(51) Int. Cl. A61K 8/37; A61K 8/44; A61K 8/9789; A61Q 17/00

(52) U.S. Cl. CPC A61K 8/37; A61K 8/44; A61K 8/9789; A61Q 17/00; A61K 2800/30

(57) ABSTRACT

Personal care compositions and personal care compositions having one or more UV filters are disclosed herein. In one aspect, provided is a personal care composition comprising a mineral UV filter; a SPF enhancer comprising at least one of butyloctyl salicylate, ethylhexyl methoxycrylene, octyldodecyl neopentanoate, or Pongamia pinnata extract; and an amino acid.

### CROSS-REFERENCE TO RELATED APPLICATIONS

This application claims the benefit of priority from U.S. Patent Application No. 63/586,691, filed Sep. 29, 2023, titled Personal Care Compositions, the content of which is hereby incorporated herein in its entirety for all purposes.

### BACKGROUND

The negative effects of exposure to ultraviolet (“UV”) light are well known. Prolonged exposure to sunlight causes damage such as sunburn to the skin and hair. Exposure to UV light at certain wavelengths, from about 290 nm to about 400 nm, for example, may lead to long-term damage as well as serious conditions such as skin cancer.

For example, ultraviolet radiation in the shorter wavelength UVB region of 290 to 320 nm is considered significant in causing burning. Accordingly, protection against UVB radiation has traditionally been a primary target of sunscreen formulations. Relatively recently there has also been a desire for further protection against UVA radiation to reduce long-term skin deterioration. Generally, UV light contributes to aging by causing free radicals, such as O⁻, OH, superoxide anion, nitric oxide, and hydrogen radicals, to form in the skin. Free radicals attack DNA, membrane lipids, and proteins, generating carbon radicals. These in turn react with oxygen to produce a peroxyl radical that can attack adjacent fatty acids to generate new carbon radicals. The end result is a loss of skin elasticity and the appearance of wrinkles.

More importantly, both the UVA and UVB wavebands have been identified as potential factors for increasing skin cancers, e.g., melanomas. Skin cancer is one of the most common cancer types and the incidence in the US is problematic. For these and other reasons, consistent use of a topical sunscreen is now strongly recommended and is widespread.

Sunscreens are subject to strict regulatory approval by various federal agencies in order to be considered “broad spectrum.” For example, to be considered a “broad spectrum” sunscreen, the current federal standard (21 C.F.R. § 201.327) requires that the mean critical wavelength must be 370 nm or greater. The critical wavelength is identified as the wavelength at which the integral of the spectral absorbance curve reaches 90 percent of the integral over the UV spectrum from 290 to 400 nm. In addition to this requirement, there are regulatory proposals that would require “broad spectrum” sunscreens to meet a UVAI/UV ratio requirement of greater than or equal to 0.70. Accordingly, there is an unmet need for broad spectrum sunscreens that can provide adequate protection from UV light and also meet the changing regulatory standards.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention is directed to personal care compositions and particularly personal care compositions having one or more UV filters. In one aspect, provided is a personal care composition comprising a mineral UV filter; a SPF enhancer comprising at least one of butyloctyl salicylate, ethylhexyl methoxycrylene, octyldodecyl neopentanoate, or Pongamia pinnata extract; and an amino acid.

In another aspect, provided is a personal care composition comprising a mineral UV filter; a SPF enhancer comprising at least one of butyloctyl salicylate, ethylhexyl methoxycrylene, octyldodecyl neopentanoate, or Pongamia pinnata extract; and a ceramide.

A list of non-limiting example personal care compositions in accordance with aspects of the invention is provided below:

In accordance with an embodiment 1, provided is a personal care composition comprising: a mineral UV filter; a SPF enhancer comprising at least one of butyloctyl salicylate, ethylhexyl methoxycrylene, octyldodecyl neopentanoate, or Pongamia pinnata extract; and an amino acid.

In accordance with an embodiment 2, provided is the personal care composition according to embodiment 1, wherein the mineral UV filter comprises a metal oxide.

In accordance with an embodiment 3, provided is the personal care composition according to embodiment 1 or embodiment 2, wherein the metal oxide is selected from zinc oxide, iron oxide, zirconium oxide, cerium oxide, and a combination of two or more thereof.

In accordance with an embodiment 4, provided is the personal care composition according to any foregoing embodiment, wherein the personal care composition is substantially free or free of titanium oxide.

In accordance with an embodiment 5, provided is the personal care composition according to any foregoing embodiment, wherein the personal care composition is substantially free or free of titanium compounds.

In accordance with an embodiment 6, provided is the personal care composition according to any foregoing embodiment, wherein the SPF enhancer comprises butyloctyl salicylate and at least one of ethylhexyl methoxycrylene or Pongamia pinnata extract.

In accordance with an embodiment 7, provided is the personal care composition according to any foregoing embodiment, wherein the amino acid comprises a plurality of amino acids selected from glycine, arginine, taurine, histidine, tyrosine, tryptophan, lysine, serine, citrulline, ornithine, creatine, diaminobutanoic acid, diaminoproprionic acid, alanine, aminobutyrate, asparagine, cysteine, cystine, glutamine, hydroxyproline, isoleucine, leucine, methionine, phenylalanine, proline, serine, taurine, threonine, tryptophan, tyrosine, valine, a salt thereof, and a combination of two or more thereof.

In accordance with an embodiment 8, provided is the personal care composition according to embodiment 7, wherein the plurality of amino acids is selected from glycine, arginine, taurine, histidine, tyrosine, tryptophan, a salt thereof, and a combination of two or more thereof.

In accordance with an embodiment 9, provided is the personal care composition according to embodiment 7 or embodiment 8, wherein the plurality of amino acids comprises glycine and/or a salt thereof, arginine and/or salt thereof, and taurine and/or a salt thereof.

In accordance with an embodiment 10, provided is the personal care composition according to any foregoing embodiment, wherein the personal care composition further comprises a ceramide.

In accordance with an embodiment 11, provided is a personal care composition comprising: a mineral UV filter; a SPF enhancer comprising at least one of butyloctyl salicylate, ethylhexyl methoxycrylene, octyldodecyl neopentanoate, or Pongamia pinnata extract; and a ceramide.

In accordance with an embodiment 12, provided is the personal care composition according to embodiment 11, wherein the mineral UV filter comprises a metal oxide.

In accordance with an embodiment 13, provided is the personal care composition according to embodiment 11 or embodiment 12, wherein the metal oxide is selected from zinc oxide, iron oxide, zirconium oxide, cerium oxide, and a combination of two or more thereof.

In accordance with an embodiment 14, provided is the personal care composition according to one of embodiment 11 to embodiment 13, wherein the personal care composition is substantially free or free of titanium oxide.

In accordance with an embodiment 15, provided is the personal care composition according to one of embodiment 11 to embodiment 14, wherein the personal care composition is substantially free or free of titanium compounds.

In accordance with an embodiment 16, provided is the personal care composition according to one of embodiment 11 to embodiment 15, wherein the SPF enhancer comprises butyloctyl salicylate and at least one of ethylhexyl methoxycrylene or Pongamia pinnata extract.

In accordance with an embodiment 17, provided is the personal care composition according to one of embodiment 11 to embodiment 16, wherein the ceramide comprises ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, ceramide NS dilaurate, or a combination of two or more thereof.

In accordance with an embodiment 18, provided is the personal care composition according to one of embodiment 11 to embodiment 17 further comprising an amino acid.

In accordance with an embodiment 19, provided is the personal care composition according to embodiment 18, wherein the amino acid comprises a plurality of amino acids selected from glycine, arginine, taurine, histidine, tyrosine, tryptophan, lysine, serine, citrulline, ornithine, creatine, diaminobutanoic acid, diaminoproprionic acid, alanine, aminobutyrate, asparagine, cysteine, cystine, glutamine, hydroxyproline, isoleucine, leucine, methionine, phenylalanine, proline, serine, taurine, threonine, tryptophan, tyrosine, valine, a salt thereof, and a combination of two or more thereof.

In accordance with an embodiment 20, provided is the personal care composition according to embodiment 18 or embodiment 19, wherein the plurality of amino acids is selected from glycine, arginine, taurine, histidine, tyrosine, tryptophan, a salt thereof, and a combination of two or more thereof.

In accordance with an embodiment 21, provided is the personal care composition according to one of embodiment 18 to embodiment 20, wherein the plurality of amino acids comprises glycine and/or a salt thereof, arginine and/or salt thereof, and taurine and/or a salt thereof.

In accordance with an embodiment 22, provided is the personal care composition according to embodiment 21, wherein the personal care composition has a weight ratio of glycine and/or a salt thereof to arginine and/or a salt thereof to taurine and/or a salt thereof of about 1:24:45 to about 1:45:85.

In accordance with an embodiment 23, provided is a personal care composition comprising: a mineral UV filter; a SPF enhancer comprising at least one of butyloctyl salicylate, ethylhexyl methoxycrylene, octyldodecyl neopentanoate, or Pongamia pinnata extract; an amino acid; and a ceramide.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is a bar graph of KI67 expression after UV exposure and application of a comparative composition and a non-limiting exemplary composition according to aspects of the invention;

FIG. 2 is a bar graph of IL-la expression after UV exposure and application of a comparative composition and a non-limiting exemplary composition according to aspects of the invention; and

FIG. 3 is a bar graph of CCNA2, KI67, COL7A1, and HAS3 expression after UV exposure and application of a comparative composition and a non-limiting exemplary composition according to aspects of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “∘” refers to a degree, such as a temperature degree or a degree of an angle. The symbols “hr”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

“Volatile”, as used herein, means having a flash point of less than about 100° C. “Non-volatile”, as used herein, means having a flash point of greater than about 100° C.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

Any member in a list of species that is used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that defines or exemplifies the genus is open, and it is given that other species may exist that define or exemplify the genus just as well as, or better than, any other species listed.

The phrases, “a mixture thereof,” “a combination thereof,” or “a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both a humectant and a polyol. If a particular personal care composition includes and/or recites both a humectant and a polyol, a compound that may be characterized as both a humectant and a polyol, such as glycerin, will serve only as either the humectant or the polyol—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention is directed to personal care compositions and particularly personal care compositions having one or more UV filters. For instance, in one aspect, provided is a personal care composition comprising a mineral UV filter; a SPF enhancer comprising at least one of butyloctyl salicylate, ethylhexyl methoxycrylene, octyldodecyl neopentanoate, or Pongamia pinnata extract; and an amino acid.

The personal care compositions may achieve a SPF value of 40 or more in some embodiments. For instance, the personal care composition may have a SPF value of about 44 or more, about 47 or more, about 49 or more, about 51 or more, about 53 or more, about 56 or more, about 60 or more, about 62 or more, about 64 or more, about 65 or more, about 67 or more, about 69 or more, about 70 or more, about 72 or more, about 75 or more, about 78 or more, or about 80 or more. Additionally or alternatively, the personal care composition may have a UVAI/UV ratio greater than or equal to 70. In certain embodiments, the personal care composition has a UVAI/UV ratio of about 72 or more, about 74 or more, about 76 or more, about 78 or more, about 80 or more, or about 82 or more.

In some embodiments, the personal care composition is formulated to have a reduced amount, be substantially free of, or be free of titanium dioxide. For instance, in some embodiments, the personal care composition comprises about 3 wt. % or less, about 2 wt. % or less, about 1 wt. % or less, about 0.5 wt. % or less, about 0.1 wt. % or less, about 0.05 wt. % or less, or about 0.01 wt. % or less of titanium dioxide, based on the total weight of the personal care composition. In at least one embodiment, the personal care composition contains about 0 wt. % or 0 wt. % of titanium dioxide, based on the total weight of the personal care composition.

Additionally or alternatively, the personal care compositions may be formulated to have a reduced amount, be substantially free of, or be free of titanium compounds. For instance, in some embodiments, the personal care composition comprises about 3 wt. % or less, about 2 wt. % or less, about 1 wt. % or less, about 0.5 wt. % or less, about 0.1 wt. % or less, about 0.05 wt. % or less, or about 0.01 wt. % or less of titanium compounds, based on the total weight of the personal care composition. In at least one embodiment, the personal care composition contains about 0 wt. % or 0 wt. % of titanium compounds, based on the total weight of the personal care composition.

Suitable components, such as those listed below, may be included or excluded from the formulations for the personal care compositions depending on the specific combination of other ingredients, the form of the personal care composition, and/or the use of such personal care composition. For example, the personal care composition may include a hydrophilic phase and a hydrophobic phase when the personal care composition is in the form of an emulsion or a two-phase composition. In some preferred embodiments, the personal care composition is in the form of a skin care composition, such as a sunscreen composition. Preferably, the personal care composition is a sunscreen composition.

The personal care composition typically comprises one or more mineral UV filter(s), preferably in an effective amount. In some instance, the amount of mineral UV filter(s) in the personal care composition is from 5 to about 25 wt. %, based on the total weight of the personal care composition. For example, the mineral UV filter(s) may be present in the personal care composition in an amount from about 5 to about 25 wt. %, about 7 to about 25 wt. %, about 9 to about 25 wt. %, about 11 to about 25 wt. %, about 13 to about 25 wt. %, about 15 to about 25 wt. %, about 18 to about 25 wt. %; from about 5 to about 21 wt. %, about 7 to about 21 wt. %, about 9 to about 21 wt. %, about 11 to about 21 wt. %, about 13 to about 21 wt. %, about 15 to about 21 wt. %, about 18 to about 21 wt. %; from about 5 to about 18 wt. %, about 7 to about 18 wt. %, about 9 to about 15 wt. %, about 11 to about 18 wt. %, about 13 to about 18 wt. %, about 15 to about 18 wt. %; from about 5 to about 15 wt. %, about 7 to about 15 wt. %, about 9 to about 15 wt. %, about 11 to about 15 wt. %, about 13 to about 15 wt. %; from about 5 to about 13 wt. %, about 7 to about 13 wt. %, about 9 to about 13 wt. %, about 11 to about 13 wt. %; from about 5 to about 11 wt. %, about 7 to about 11 wt. %, about 9 to about 11 wt. %; from about 5 to about 9 wt. %, about 7 to about 9 wt. %; from about 5 to about 8 wt. %, about 7 to about 8 wt. %, about 5 to about 7 wt. %, or any range or subrange thereof, based on the total weight of the personal care composition.

The mineral UV filters may comprise metal oxides, including titanium oxide (amorphous or crystallized in rutile and/or anatase form), iron oxide, zinc oxide, zirconium oxide, and/or cerium oxide. The metal oxides may comprise treated or untreated metal oxides. In some preferred embodiments, the mineral UV filter comprises or is selected from the group consisting of iron oxide, zinc oxide, zirconium oxide, cerium oxide, and a combination thereof. For example, the mineral UV filter may include or consisting of one or more of iron oxide(s), zinc oxide(s), zirconium oxide(s), or a combination of two or more thereof. In at least one embodiment, the personal care composition includes one or more zinc oxide(s). The personal care composition, in some instances, may consist of mineral UV filters selected from zinc oxides.

The metal oxide may have a mean particle size of about 5 nm to about 30 μm. In some instances, the mean particle size of the metal oxide may be about 5 nm to about 25 μm, e.g., preferably, about 10 nm to about 10 μm, or about 15 nm to about 5 μm. In further instances, the metal oxide may a preferable mean particle size of about 5 nm to about 100 nm, about 5 nm to about 75 nm, or about 10 nm to 50 nm. Larger particles sizes may also be useful, such as, e.g., from about 1 μm to about 25 μm, about 5 μm to about 20 μm, or about 10 μm to about 15 μm. The treated metal oxides may have a coating formed thereon.

Treated metal oxides typically have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina, or glycerol.

In certain embodiments, mineral UV filter includes one or more zinc oxides, wherein at least one zinc oxide is a treated and/or coated zinc oxide. Examples of treated and/or coated zinc oxides include zinc oxides coated with silica and polymethylhydrogenosiloxane, zinc oxide coated with triethoxycaprylysilane, zinc oxides coated with polymethylhydrogenosiloxane, zinc oxides coated with perfluoroalkyl phosphate and copolymer based on perfluoroalkylethyl as a dispersion in cyclopentasiloxane, and zinc oxides coated with silica and polymethylsilsesquioxane.

Although the personal care composition may have a reduced amount, be substantially free of, or free of titanium compounds, in some embodiments the personal care composition may include a titanium oxide, such as a treated and/or coated titanium oxide. Examples of treated and/or coated titanium oxides include those treated with: silica and alumina; alumina and aluminium stearate; iron oxides and iron stearate; silica, alumina and silicone; sodium hexametaphosphate; octyltrimethoxysilane; alumina and stearic acid; alumina and glycerol; alumina and silicone; and combinations thereof.

In certain embodiments, the majority of the mineral UV filters in the personal care composition is treated and/or coated. For instance, the personal care composition, in some preferred embodiments, may have about 50 wt. % or more, about 60 wt. % or more, about 65 wt. % or more, about 70 wt. % or more, about 75 wt. % or more, about 80 wt. % or more, about 90 wt. % or more, about 95 wt. % or more, about 97 wt. % or more, about 98 wt. % or more, about 99 wt. % or more, about 100 wt. %, or any range formed therefrom, relative to the total amount of mineral UV filters, of the mineral UV filters being treated and/or coated. For example, the amount of treated and/or coated mineral UV filter(s) present in the personal care composition may be from about 5 to about 25 wt. %, about 7 to about 25 wt. %, about 9 to about 25 wt. %, about 11 to about 25 wt. %, about 13 to about 25 wt. %, about 15 to about 25 wt. %, about 18 to about 25 wt. %; from about 5 to about 21 wt. %, about 7 to about 21 wt. %, about 9 to about 21 wt. %, about 11 to about 21 wt. %, about 13 to about 21 wt. %, about 15 to about 21 wt. %, about 18 to about 21 wt. %; from about 5 to about 18 wt. %, about 7 to about 18 wt. %, about 9 to about 15 wt. %, about 11 to about 18 wt. %, about 13 to about 18 wt. %, about 15 to about 18 wt. %; from about 5 to about 15 wt. %, about 7 to about 15 wt. %, about 9 to about 15 wt. %, about 11 to about 15 wt. %, about 13 to about 15 wt. %; from about 5 to about 13 wt. %, about 7 to about 13 wt. %, about 9 to about 13 wt. %, about 11 to about 13 wt. %; from about 5 to about 11 wt. %, about 7 to about 11 wt. %, about 9 to about 11 wt. %; from about 5 to about 9 wt. %, about 7 to about 9 wt. %; from about 5 to about 8 wt. %, about 7 to about 8 wt. %, about 5 to about 7 wt. %, or any range or subrange thereof, based on the total weight of the personal care composition.

In further embodiments, however, the personal care composition may include treated and/or coated metal oxides in an amount from about 0.01 to about 3 wt. %, based on the total weight of the personal care composition. For instance, the amount of treated and/or coated metal oxides in the personal care composition may be from about 0.01 to about 3 wt. %, about 0.01 to about 2 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %; from about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.2 to about 1 wt. %, about 0.2 to about 0.8 wt. %, about 0.2 to about 0.6 wt. %, about 0.2 to about 0.4 wt. %; from about 0.5 to about 1 wt. %, about 0.5 to about 0.8 wt. %, about 0.5 to about 0.6 wt. %; from about 0.8 to about 1 wt. %, about 0.8 to about 1.5 wt. %, about 0.8 to about 2 wt. %, or any range or subrange thereof, based on the total weight of the personal care composition. In certain embodiments, the amount of treated and/or coated metal oxides in the personal care composition is about 0.01 wt. %, about 0.05 wt. %, about 0.1 wt. %, about 0.15 wt. %, about 0.2 wt. %, or any range or subrange thereof, based on the total weight of the personal care composition

The personal care compositions also typically include one or more SPF enhancer(s). The one or more SPF enhancer(s) may be present in the personal care composition in an amount from about 0.5 to about 10 wt. %, relative to the total weight of the personal care composition. For instance, the personal care composition may comprise the SPF enhancer(s) in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the personal care composition.

Preferably, the one or more SPF enhancer(s) comprises at least one of butyloctyl salicylate, ethylhexyl methoxycrylene, octyldodecyl neopentanoate, or Pongamia pinnata extract. In some embodiments, the personal care composition comprises at least two, at least three, or at least four of butyloctyl salicylate, ethylhexyl methoxycrylene, octyldodecyl neopentanoate, Pongamia pinnata extract, or combinations thereof. For example, the SPF enhancer may comprise butyloctyl salicylate and at least one of ethylhexyl methoxycrylene, octyldodecyl neopentanoate, or Pongamia pinnata extract. In certain embodiments, the SPF enhancer includes butyloctyl salicylate and at least one of ethylhexyl methoxycrylene or Pongamia pinnata extract. In further embodiments, the one or more SPF enhancer(s) are selected from the group consisting of butyloctyl salicylate, ethylhexyl methoxycrylene, octyldodecyl neopentanoate, Pongamia pinnata extract, and a combination of two or more thereof.

Additional SPF enhancers may be included in the personal care composition. The SPF enhancer(s) may be selected from photostabilizers and/or antioxidants. Non-limiting examples of photostabilizers include ethylhexyl triazone, dioctyl butamido triazone, benzylidene malonate polysiloxane, bis benzoxazoylphenyl ethylhexylimino triazine, drometrizole trisiloxane, methylene bis-benzotriazolyl tetramethylbutylphenol, bis-ethylhexyloxyphenol methoxyphenyltriazine, butyloctyl salicylate, alpha-cyanodiphenylacrylate (e.g., ethylhexyl methoxycrylene or 2-ethylhexyl 2-cyano-3-(4-methoxyphenyl)-3-phenylpropenoate), isopentyl 4-methoxycinnamate, diethylhexyl syringylidenemalonate, diethylhexyl 2,6-naphthalate, and octocrylene. In some embodiments, the photostabilizer comprises dioctyl butamido triazone, benzylidene malonate polysiloxane, butyloctyl salicylate, alpha-cyanodiphenylacrylate (e.g., ethylhexyl methoxycrylene or 2-ethylhexyl 2-cyano-3-(4-methoxyphenyl)-3-phenylpropenoate), isopentyl 4-methoxycinnamate, diethylhexyl syringylidenemalonate, diethylhexyl 2,6-naphthalate, octocrylene, or a combination of two or more thereof.

The amount of photostabilizers present in the personal care composition may be from about 0.5 to about 10 wt. %, relative to the total weight of the personal care composition. For instance, the personal care composition may have an amount of photostabilizer(s) ranging from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the personal care composition.

The personal care composition may include one or more SPF enhancer that is an antioxidant. The antioxidant may comprise or be selected from the group consisting of Angelica archangelica, Arnica montana, Camellia sinensis, Cupressus semper, Coffee arabica, Polygonatum multiflorum, Pongamia pinnata extract, Rosmarinus officinalis, Evernia fufuracea, Evernia prunastri, Aventa sativa, vitamin A, vitamin E, peptides, flavones, flavonols, and/or a combination of two or more thereof.

In some embodiments, the antioxidant(s) may be present in an amount from about 0.1 to about 5 wt. %, relative to the total weight of the personal care composition. For instance, the personal care composition may, in some cases, include one or more antioxidant(s) in an amount from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, or any range or subrange thereof, based on the total weight of the personal care. The total amount of antioxidant(s) in the personal care composition may in certain embodiments be from about 0.01 to about 1 wt. %, about 0.01 to about 0.8 wt. %, about 0.01 to about 0.5 wt. %, about 0.01 to about 0.3 wt. %, about 0.01 to about 0.2 wt. %, about 0.01 to about 0.1 wt. %; from about 0.05 to about 1 wt. %, about 0.05 to about 0.8 wt. %, about 0.05 to about 0.5 wt. %, about 0.05 to about 0.3 wt. %, about 0.05 to about 0.2 wt. %, about 0.05 to about 0.1 wt. %; from about 0.1 to about 1 wt. %, about 0.1 to about 0.8 wt. %, about 0.1 to about 0.5 wt. %, about 0.1 to about 0.3 wt. %, about 0.1 to about 0.2 wt. %, about 0.1 to about 0.1 wt. %, or any range or subrange thereof, based on the total weight of the personal care composition.

The personal care compositions typically comprise at least one amino acid and/or at least one ceramide. For example, in certain preferred embodiments, the personal care composition comprises one or more amino acid. The one or more amino acid may be present in an effective amount. In some embodiments, the one or more amino acids are present in an amount from about 0.1 to about 5 wt. %, based on the total weight of the personal care composition. For example, the personal care composition may include one or more amino acids(s) in an amount from about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, or any range or subrange thereof, based on the total weight of the personal care. The total amount of amino acid(s) in the personal care composition may in certain embodiments be from about 0.01 to about 1 wt. %, about 0.01 to about 0.8 wt. %, about 0.01 to about 0.5 wt. %, about 0.01 to about 0.3 wt. %, about 0.01 to about 0.2 wt. %, about 0.01 to about 0.1 wt. %; from about 0.05 to about 1 wt. %, about 0.05 to about 0.8 wt. %, about 0.05 to about 0.5 wt. %, about 0.05 to about 0.3 wt. %, about 0.05 to about 0.2 wt. %, about 0.05 to about 0.1 wt. %; from about 0.1 to about 1 wt. %, about 0.1 to about 0.8 wt. %, about 0.1 to about 0.5 wt. %, about 0.1 to about 0.3 wt. %, about 0.1 to about 0.2 wt. %, about 0.1 to about 0.1 wt. %, or any range or subrange thereof, based on the total weight of the personal care composition.

The one or more amino acids may comprise or consist of arginine, lysine, serine, citrulline, ornithine, creatine, histidine, diaminobutanoic acid, diaminoproprionic acid, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, a salt thereof, and/or a combination of two or more thereof. The personal care composition may include a plurality of amino acids in some instances. For instance, the personal care composition may include a plurality of amino acids comprising or selected from the group consisting of taurine, arginine, glycine, lysine, ornithine, proline, and/or a combination of two or more thereof. In some preferred embodiments, the plurality of amino acids comprise or are selected from the group consisting of glycine, arginine, taurine, histidine, tyrosine, tryptophan, a salt thereof, and/or a combination of two or more thereof. In certain embodiments, the plurality of amino acids comprises taurine and/or a salt thereof, arginine and/or a salt thereof, and glycine and/or a salt thereof.

The personal care composition may be formulated in certain desirable embodiments to have a weight ratio of taurine and/or a salt thereof to arginine and/or a salt thereof to glycine and/or a salt thereof (taurine:arginine:glycine) of (1.5-69):(0.5-40):(0.5-1.5). For instance, the personal care composition may preferably be formulated to have a weight ratio of taurine and/or a salt thereof to arginine and/or a salt thereof to glycine and/or a salt thereof (taurine:arginine:glycine) from about 85:45:1 to about 45:24:1. In some preferred embodiments, the personal care composition has a weight ratio of taurine and/or a salt thereof to arginine and/or a salt thereof to glycine and/or a salt thereof from about 80:42:1 to about 50:30:1; from about 75:40:1 to about 55:29:1; from about 70:37:1 to about 60:31:1, or any range or subrange thereof. In at least one embodiment, the weight ratio of taurine and/or a salt thereof to arginine and/or a salt thereof to glycine and/or a salt thereof (taurine:arginine:glycine) are in a weight ratio of about 65:34:1.

One or more ceramides may be present in the personal care composition in an effective amount. In some embodiments, the ceramide(s) is present amount from about 0.01 to about 2 wt. %, based on the total weight of the personal care composition. For instance, the total amount of ceramide(s) in the personal care composition may be about 0.01 to about 1.5 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %; from about 0.05 to about 1.5 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 1.5 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.3 to about 1.5 wt. %, about 0.3 to about 1 wt. %, about 0.3 to about 0.8 wt. %, about 0.3 to about 0.6 wt. %, about 0.3 to about 0.5 wt. %; from about 0.5 to about 1.5 wt. %, about 0.5 to about 1 wt. %, about 0.5 to about 0.8 wt. %, about 0.5 to about 0.6 wt. %; from about 0.8 to about 1.5 wt. %, about 0.8 to about 1 wt. %, or any range or subrange thereof, based on the total weight of the personal care composition.

Ceramides are a family of waxy lipid molecules that are composed of sphingosine and a fatty acid. Non-limiting examples of ceramides include ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, and ceramide NS dilaurate. In some instances, the personal care compositions include ceramide 3, optionally in combination with one or more other ceramides. In other instances, the personal care compositions include ceramide EOP, optionally in combination with one or more other ceramides. The personal care compositions may include two, three, or more ceramides. For example, the ceramide 3 and ceramide EOP (or any other combination of two or more ceramides) could be included in the personal care compositions.

In at least one preferred embodiment, the personal care composition comprises or consists of ceramides selected from the group of ceramide EOP, ceramide NP, ceramide AP, and/or a combination of two or more thereof. For instance, the personal care composition may comprise ceramide EOP, ceramide NP, and Ceramide AP.

The personal care composition may, optionally, include an organic UV filter. In some embodiments, however, the personal care composition may exclude certain organic UV filters. Examples of organic UV filters that may be incorporated into or excluded from the personal care composition include anthranilic compounds; dibenzoylmethane compounds; cinnamic compounds; salicylic compounds; camphor compounds; benzophenone compounds; y,y-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenyl benzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

The personal care composition may, in some embodiments, include one or more oils, such as those selected from silicone oils, fluoro oils, hydrocarbon-based oils, etc. The term “oil” means any fatty substance which is in liquid form at room temperature (20-25° C.) and at atmospheric pressure (760 mmHg). Often, at least one of the oils in the personal care composition is part of an oily phase. An “oily phase” is a phase comprising at least one oil that may include additional liposoluble and lipophilic ingredients and the fatty substances. Oil that is suitable for use herein may be volatile or non-volatile. The term “volatile oil” relates to an oil that is capable of evaporating on contact with the skin or a keratin fiber in less than one hour, at room temperature and atmospheric pressure. The volatile oil(s) are generally liquid at room temperature and have a non-zero vapor pressure, at room temperature and atmospheric pressure, ranging in particular from 0.13 Pa to 40 000 Pa (10-3 to 300 mmHg). The term “non-volatile oil” relates to an oil that remains on the skin or the keratin fiber, at room temperature and atmospheric pressure, for at least several hours and which in particular has a vapor pressure of less than 10-3 mmHg (0.13 Pa).

The term “silicone oil” relates to an oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil or a synthetic hydrocarbon-based oil. Further, suitable oil may be a mineral hydrocarbon-based oil, a plant hydrocarbon-based oil, or a synthetic hydrocarbon-based oil. Non-limiting examples of silicone oils include dimethicone, cyclomethicone, polysilicone-11, phenyl trimethicone, trimethylsilylamodimethicone, and stearoxytrimethylsilane. In some cases, the personal care composition includes dimethicone, and optionally additional oils, including additional silicone oils. Typically, the one or more silicone oil is a non-volatile silicon oil. In some embodiments, the silicone oil is dimethicone, polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. In at least one embodiment, the personal care composition comprises dimethicone having a viscosity of about 1 to about 10 cSt, e.g., about 5 cSt.

The personal care compositions described herein may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the personal care compositions include a synthetic oil selected from the group consisting of hydrogenated polyisobutene, hydrogenated polydecene, and a combination thereof.

The oil may be a non-volatile and hydrocarbon-based oil, such as hydrocarbon-based oils of plant origin, such as glyceride triesters, which are generally triesters of fatty acids and of glycerol, the fatty acids of which can have varied chain lengths from C₄ to C₂₄, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil. In certain instances, the non-volatile hydrocarbon-based oils are chosen from glyceride triesters and in particular to caprylic/capric acid triglycerides, synthetic esters and in particular isononyl isononanoate, oleyl erucate, C₁₂-C₁₅ alkyl benzoate, 2-ethylphenyl benzoate and fatty alcohols, such as octyldodecanol.

The personal care composition may include a cosmetically acceptable carrier other than water. The phrase “cosmetically acceptable” means that the material is compatible with skin and hair. For example, “cosmetically acceptable carrier” means a carrier that is compatible with skin and hair, and is acceptable for application to the body.

Non-limiting examples of cosmetically acceptable carriers other than water include glycerin, C₁₋₄ alcohols, organic solvents, fatty alcohols, fatty ethers, fatty esters, polyols, glycols, vegetable oils, mineral oils, liposomes, laminar lipid materials, water, or any combinations thereof. As examples of organic solvents, non-limiting mentions can be made of monoalcohols, such as ethyl alcohol, isopropyl alcohol, propyl alcohol, benzyl alcohol, and phenylethyl alcohol.

Non-limiting examples of polyol(s) include those chosen from glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Polyols that may be included in the personal care composition, in certain instances, include ethylene glycol, propylene glycol, butylene glycol, hexylene glycol, glycerin, diglycerin, diethylene glycol, dipropylene glycol, and mixtures thereof. In some cases, the personal care composition comprises glycerin, and optionally one or more polyols other than glycerin. Non-limiting examples of polyols that may, optionally, be included in the personal care include alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, or a combination of two or more thereof. In at least one embodiment, the personal care composition includes one or more polyol(s) comprising panthenol, pantothenic acid, or a combination thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, or a combination of two or more thereof. In certain embodiments, the personal care composition may include polyol(s) selected from glycerin (preferably, vegetable refined glycerin), butylene glycol, and 1,3-propanediol, polyglutamic acid, saccharide isomerate, and combinations thereof. In some embodiments, the polyol is a polyethylene glycol, such as polyethylene glycol 600.

Additionally or alternatively, the personal care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof.

The personal care composition may include one or more humectant(s) e.g., in an amount ranging from about 0.01 to about 5 wt. %, based on the total weight of the personal care composition. For example, the one or more humectant(s) may be present in a total amount from about 0.01 to about 4 wt. %, about 0.01 to about 3 wt. %, about 0.01 to about 2 wt. %, about 0.01 to about 1 wt. %, about 0.01 to about 0.75 wt. %, about 0.01 to about 0.5 wt. %, about 0.01 to about 0.25 wt. %, about 0.01 to about 0.1 wt. %; from about 0.05 to about 5 wt. %, about 0.05 to about 3 wt. %, about 0.05 to about 2 wt. %, about 0.05 to about 1 wt. %, about 0.05 to about 0.75 wt. %, about 0.05 to about 0.5 wt. %, about 0.05 to about 0.25 wt. %, about 0.05 to about 0.1 wt. %; from about 0.1 to about 5 wt. %, about 0.1 to about 3 wt. %, about 0.1 to about 2 wt. %, about 0.1 to about 1 wt. %, about 0.1 to about 0.75 wt. %, about 0.1 to about 0.5 wt. %, about 0.1 to about 0.25 wt. %; from about 0.15 to about 5 wt. %, about 0.15 to about 3 wt. %, about 0.15 to about 2 wt. %, about 0.15 to about 1 wt. %, about 0.15 to about 0.75 wt. %, about 0.15 to about 0.5 wt. %, about 0.15 to about 0.25 wt. %; from about 0.4 to about 5 wt. %, about 0.4 to about 3 wt. %, about 0.4 to about 2 wt. %, about 0.4 to about 1 wt. %, about 0.4 to about 0.75 wt. %; from about 0.8 to about 5 wt. %, about 0.8 to about 3 wt. %, about 0.8 to about 2 wt. %, about 0.8 to about 1 wt. %; from about 1.2 to about 5 wt. %, about 1.2 to about 3 wt. %, about 1.2 to about 2 wt. %; from about 1.6 to about 5 wt. %, about 1.6 to about 3 wt. %, about 1.6 to about 2 wt. %; from about 2 to about 7 wt. %, about 2 to about 5 wt. %, about 2 to about 3 wt. %, or any range or subrange thereof, based on the total weight of the personal care composition.

Examples of humectants that may be incorporated into the personal care composition include, but are not limited to, polyhydric alcohols such as glycerin, Centella asiatica leaf extract, saccharide isomerate butylene glycol, 1,3-propanediol, sorbitol, xylitol or low molecular weight polyethylene glycols (PEGs), polyoxyethylenes or combinations thereof. In some embodiments, the humectant agents are sorbitol (preferably, non-crystal sorbitol), butylene glycol,1,3-propanediol, glycerin (e.g., vegetable refined glycerin), polyoxyethylene glycol or combinations thereof. In at least one embodiment, the humectants are chosen from glycerin, Centella asiatica leaf extract, butylene glycol and 1,3-propanediol. Typically, the humectant is vegetable refined glycerin. Other suitable humectants include, but are not limited to, polyglutamic acid, saccharide isomerate or combinations thereof. In at least one instance, the humectant is saccharide isomerate. Saccharide isomerate can be commercially obtained from, e.g., DSM under the trademark of Pentavitin™. Additional examples of humectants include glycerol and its derivatives, urea and its derivatives, sodium pidolate, xylitol, serine, sodium lactate, ectoin and its derivatives, chitosan and its derivatives, collagen, plankton, an extract of Imperata cylindra, beta-glucan, and/or rose hip oil. In certain embodiments, the personal care composition includes a humectant selected from sorbitol, Centella asiatica leaf extract, butylene glycol,1,3-propanediol, glycerin, polyoxyethylene glycol, polyglutamic acid, saccharide isomerate, or a combination of two or more thereof.

The personal care composition typically includes an amount of water. The amount of water present in the personal care composition may be from about 10 to about 90 wt. %, about 15 to about 90 wt. %, about 20 to about 90 wt. %, about 30 to about 90 wt. %, about 40 to about 90 wt. %, about 50 to about 90 wt. %, about 60 to about 90 wt. %, about 70 to about 90 wt. %, about 80 to about 90 wt. %; about 10 to about 80 wt. %, about 15 to about 80 wt. %, about 20 to about 80 wt. %, about 30 to about 80 wt. %, about 40 to about 80 wt. %, about 50 to about 80 wt. %, about 60 to about 80 wt. %, about 70 to about 80 wt. %; about 10 to about 70 wt. %, about 15 to about 70 wt. %, about 20 to about 70 wt. %, about 30 to about 70 wt. %, about 40 to about 70 wt. %, about 50 to about 70 wt. %, about 60 to about 70 wt. %; about 10 to about 60 wt. %, about 15 to about 60 wt. %, about 20 to about 60 wt. %, about 30 to about 60 wt. %, about 40 to about 60 wt. %, about 50 to about 60 wt. %; about 10 to about 50 wt. %, about 15 to about 50 wt. %, about 20 to about 50 wt. %, about 30 to about 50 wt. %, about 40 to about 50 wt. %, including any ranges and subranges thereof, based on the total weight of the personal care composition.

The personal care composition may optionally include one or more thickening agent(s). The amount of thickening agent in the personal care composition, if present, may be from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the personal care composition.

The thickener(s) may be chosen from carboxylic acid polymers; crosslinked polyacrylate polymers; polyacrylamide polymers; gums, polysaccharides, and combinations of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof.

Non-limiting examples of carboxylic acid polymers include crosslinked carboxylic acid polymers containing one or more monomers derived from acrylic acid, substituted acrylic acids, and salts and esters of these acrylic acids and the substituted acrylic acids, such as carbomers. Non-limiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and a combination of two or more thereof.

In certain cases, the personal care composition may be formulated to have thickening polymers, such as polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (e.g., carboxymethyl cellulose, methyl cellulose, methylhydroxypropyl cellulose, hydroxyethyl cellulose, hydroxypropyl cellulose, cellulose sulfate sodium salt), and/or starch derivatives (e.g., starch oxide, dialdehyde starch, dextrin, British gum, acetyl starch, starch phosphate, carboxymethyl starch, hydroxyethyl starch, hydroxypropyl starch). In some embodiments, the thickener(s) is selected from among hydroxypropyl methylcellulose, polyethylene glycols (PEGs), polyacrylic acids, cross-linked homopolymer of acrylic acid, and acrylates of C₁₀₋₃₀ alkyl acrylate crosspolymer.

The thickening agents may be chosen from xanthan gum, guar gum, biosaccharide gum, cellulose, acacia seneca gum, sclerotium gum, agarose, pechtin, gellan gum, hyaluronic acid, and a combination of two or more thereof. Additionally, the thickening agents may include polymeric thickeners selected from the group consisting of ammonium polyacryloyldimethyl taurate, ammonium acryloyldimethyltaurate/VP copolymer, acrylates copolymers, polyacrylamide, carbomer, acrylates/C10-30 alkyl acrylate crosspolymer, and a combination of two or more thereof. In at least one preferred embodiment, the personal care composition includes Hydroxyethyl Acrylate/Sodium Acryloyldimethyl Taurate Copolymer. One or more of the foregoing thickening agents may be excluded from the certain embodiments of the personal care composition.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

In certain embodiments, the personal care composition has a pH of from about 5 to about 10. In other embodiments, the personal care composition has a pH of from about 6 to about 8. In some embodiments, the personal care composition has a pH of from about 6 to about 8.5. In other embodiments, the personal care composition has a pH of from about 6.5 to about 8.5. In further embodiments, the personal care composition has a pH of from about 7 to about 8.

Additional ingredients may be present in the personal care composition. These include ingredients to thicken, preserve, emulsify, add fragrance, adjust the pH, and color. The personal care compositions may include any of the following additional ingredients in an amount of from about 0.01 to about 5 wt. %. In some instances, the amount of additional ingredients present in the personal care composition is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the personal care composition.

The personal care composition can comprise additional ingredients, such as proteins (e.g., hydrolyzed vegetable protein, hydrolyzed wheat protein, hydrolyzed milk protein, hydrolyzed silk and hydrolyzed collagen), emollients (e.g., esters, isopropyl myristate, decyl oleate, C₁₂₋₁₅ alkyl benzoate), botanicals (e.g., chamomile, aloe, rosemary), as well as preservatives, dyes, pH adjusters and chelating agents. Additional examples of vitamins that may be included in some personal care compositions include tocopherol, tocopheryl acetate, retinol, and ascorbic acid. The additional ingredients may include clays, such as magnesium silicate and/or magnesium aluminum silicate.

Additionally or alternatively, the personal care composition may comprise an additional ingredient selected from carnosine, cica (asiaticoside), hyaluronic acid and salts thereof (e.g., sodium hyaluronate), magnesium sulfate, saccharide isomerate, a keto-carotenoid (e.g., astaxanthin), nicotinamide adenine dinucleotide (NAD), and a combination of two or more thereof. the additional ingredient may include emulsifiers, such as oleth-20, oleth-3 phosphate, diethanolamine, and a combination thereof.

Non-limiting examples of fragrances and perfumes include odor compounds selected from: 7-acetyl-1,2,3,4,5,6,7,8-octahydro-1,1,6,7-tetramethylnaphthalene, α-ionone, β-ionone, γ-ionone α-isomethylionone, methylcedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl) propionaldehyde, ethylvanillin, heliotropin, hexylcinnamaldehyde, amylcinnamaldehyde, 2-methyl-2-(isopropylphenyl) propionaldehyde, coumarin, γ-decalactone, cyclopentadecanolide, 16-hydroxy-9-hexadecenoic acid lactone, 1,3,4,6,7,8-hexahydro-4,6,6,7,8,8-hexamethylcyclopenta-γ-2-benzopyran, β-naphthol methyl ether, ambroxane, dodecahydro-3a,6,6,9a-tetramethylnaphtho[2,1b]furan, cedrol, 5-(2,2,3-trimethylcyclopent-3-enyl)-3-methylpentan-2-ol, 2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, and tert-butylcyclohexyl acetate.

Other fragrances may include odor compounds selected from essential oils, resinoids and resins from a large number of sources, such as, for example, Peru balsam, olibanum resinoid, styrax, labdanum resin, nutmeg, cassia oil, benzoin resin, coriander, and lavandin.

Further suitable fragrances include odor compounds selected from phenylethyl alcohol, terpineol, linalool, linalyl acetate, geraniol, nerol, 2-(1,1-dimethylethyl)cyclo-hexanol acetate, benzyl acetate, and eugenol. The fragrances or perfumes can be used as single substances or in a mixture with one another.

The personal care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes.

The colorants may be present in an amount ranging from about 0.0001 to about 0.4 wt. %, including all percentages and subranges therebetween, based on the total weight of the personal care composition. In some embodiments, the colorants may be present in an amount ranging from about 0.0001 to about 4 wt. %, including all percentages and subranges therebetween, based on the total weight of the personal care composition. In certain embodiments, in order to prevent ingredients from separating, emulsifiers may be added.

In other embodiments, the personal composition may include any of following materials in any desired amount to achieve a desired effect in the composition (amounts that can be used in some embodiments are provided): one or more alkaline salts, for example, sodium chloride, sodium sulfate, sodium carbonate, sodium bicarbonate and/or their equivalents (0 to 5% by weight); sequestrants, for example, tetrasodium EDTA, and/or their equivalents (0 to 2% by weight); biocides, for example, Triclosan (2,4,4′-trichloro-2′-hydroxydiphenyl ether), DMDM hydantoin, formaldehyde and/or imidazolidinyl urea, and/or their equivalents (0 to 2% by weight); organic acids, for example, citric acid and/or formic acid and/or their equivalents (0 to 2% by weight); viscosity modifiers (0 to 2% by weight); fragrances and/or perfumes (0 to 5% by weight); preservatives, for example, phenoxyethanol, formaldehyde solution, parabens, pentanediol or sorbic acid (0 to 2% by weight); pearlizing agents, for example, glycol distearic esters, such as ethylene glycol distearate, but also fatty acid monoglycol esters (0 to 3% by weight); stabilizers, for example, metal salts of fatty acids, such as e.g. magnesium stearate, aluminum stearate and/or zinc stearate (0 to 2% by a weight); and dyes and pigments that are approved and suitable for cosmetic purposes.

### EXAMPLES

### Example 1

A non-limiting, example personal care composition (Ex. A) was prepared in accordance with aspects of the invention. Ex. A was in the form of a sunscreen composition having the formulation shown in Table 1.

TABLE 1

| | Ex. A |
|---|---|
| Ingredient | (wt. %) |
| Zinc Oxide and Triethoxycaprylysilane | 7.5-20 |
| Butyloctyl Salicylate | 1-10 |
| Ethylhexyl Methoxycrylene | 1-5 |
| Pongamia Pinnata Extract | 0.01-0.1 |
| Octyldodecyl Neopentanoate | 1-5 |
| Salicyloyl Phytosphingosine | 0.1-0.5 |
| Butanediol | 0.1-3 |
| Caprylic/Capric Triglyceride | 1-5 |
| Dimethicone (5 cSt) | 1-10 |
| Oleth-20 and Oleth-3 Phosphate | 0.1-3 |
| Hydroxyethyl Acrylate/Sodium | 1-5 |
| Acryloyldimethyl Taurate Copolymer | |
| Glycerin (and) Aqua (and) Centella | 0.1-0.5 |
| Asiatica Leaf Extract | |
| Ceramide EOP (and) Ceramide NP | 0.05-0.25 |
| (and) Ceramide AP (and) Behenic Acid | |
| (and) Polyglyceryl-10 Stearate (and) | |
| Polyglyceryl-6 Behenate (and) Glycerin | |
| (and) Lactic Acid | |
| Glycine | 0.01-0.1 |
| Arginine | 0.1-1 |
| Taurine | 0.5-2 |
| Tocopheryl Acetate | 0.1-0.5 |
| alpha Tocopherol | 0.05-0.25 |
| Lactic Acid and Citric Acid | 0.1-1 |
| Phenoxyethanol, Ethylhexylglycerin, | 0.5-2 |
| and Citric Acid | |
| Water | Q.S. to 100 |

### Example 2

Another non-limiting, example personal care composition (Ex. B) was prepared in accordance with an aspect of the invention. A comparative composition (Comp. 1) was also prepared. Ex. B and Comp. 1 were in the form of sunscreen compositions and had formulations as shown in Table 2.

TABLE 2

| | Ex. B | Comp. 1 |
|---|---|---|
| Ingredient | (wt. %) | (wt. %) |
| Zinc Oxide (and) | 10-15 | 10-15 |
| triethoxycaprylysilane | | |
| Butyloctyl Salicylate | 1-10 | 1-10 |
| Ethylhexylmethoxycrylene | 1-5 | 1-5 |
| Octyldodecyl Neopentanoate | 1-10 | 1-10 |
| Glycine | 0.01-0.1 | — |
| Arginine | 0.1-2 | — |
| Taurine | 1-4 | — |
| Dimethicone (2 cSt) | 2-10 | 2-10 |
| Oleth-3 Phosphate | 0.01-0.1 | 0.01-0.1 |
| Hydroxyethyl | 1-5 | 1-5 |
| Acrylate/Sodium | | |
| Acryloyldimethyl Taurate | | |
| Copolymer | | |
| Lactic Acid | 0.005-0.025 | 0.005-0.025 |
| Phenoxyethanol (and) | 0.1-3 | 0.1-3 |
| Ethylhexylglycerin | | |
| Water | Q.S. to 100 | Q.S. to 100 |

### Example 3

Ex. B and Comp. 1 were applied to in vitro skin samples and evaluated to assess the effect of the respective sunscreen compositions. Specifically, in vitro EPIDERM™ systems were obtained from MATTEK. The in vitro tissues of the EPIDERM™ system was equilibrated overnight. A 10 μL sample of Ex. B or a 10 μL sample of Comp. 1 was applied to the surface of the in vitro tissue. The in vitro tissue was then kept at a temperature of 37° C. for one hour.

The in vitro tissues were then irradiated with UV light at 20 J/cm² for one hour using a solar simulator. After UV light irradiation, the tissue culture media was replaced by a fresh media, followed by 24 hour of incubation at a temperature of 37° C. Additional tissues were prepared according to similar procures, but did not receive either Ex. B or Comp. 1 and were not subject to irradiation to serve a control.

The efficacy of Ex. B and Comp. 1 was then assessed. Specifically, the in vitro skin samples were evaluated to assess the amount of IL-la expression, KI67 expression, CCNA2 expression, COL7A1 expression, and HAS3 expression. Culture media was collected for IL-la ELISA and RNA was extracted from tissues. RNA was converted to cDNA for QPCR analysis for gene expression to evaluate expression of KI67, CCNA2, COL7A1, and HAS3.

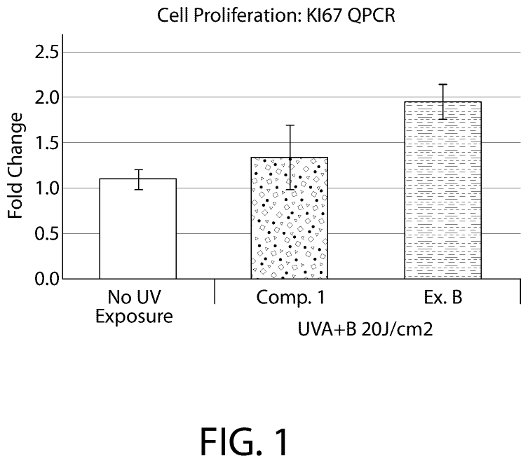

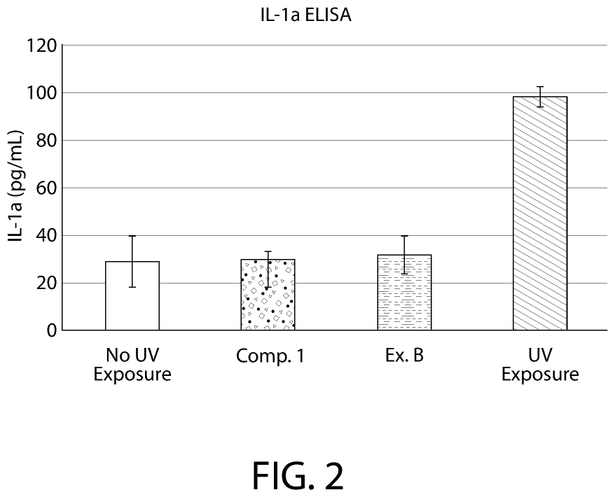

A summary of the KI67 expression in the in vitro skin samples after exposure to the UV light is shown in FIG. 1. KI67 is a marker for cell proliferation. With UV light irradiation, keratinocytes halt cell proliferation; therefore, KI67 expression is downregulated. An increase in KI67 expression indicates protection from UV light as well improved cell turnover for keratinocytes (epidermis). A summary of the IL-la expression in the in vitro skin samples after exposure to the UV light is shown in FIG. 2.

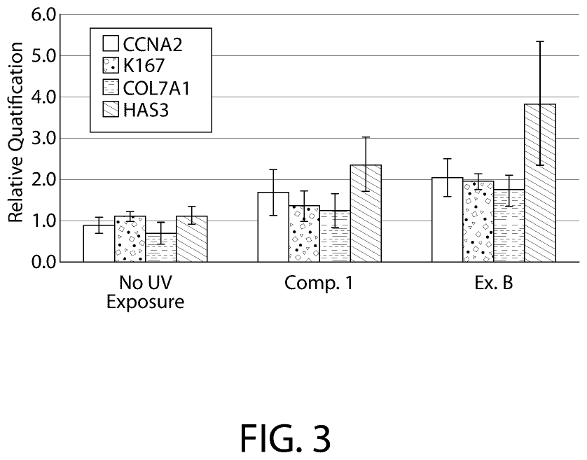

A summary of the CCNA2 expression, COL7A1 expression, MKI67 expression, and HAS3 expression in the in vitro skin samples after irradiation with the UV light is shown in FIG. 3. Hyaluronan synthase 3 (HAS3) promotes hyaluronan; thus, higher HAS3 expression indicates increased hyaluronan expression, resulting in improved hydration. Collagen Type VII alpha 1 chain (COL7A1) is collagen expressed in the dermal epidermal junction. An increased COL7A1 expression indicates improved anchoring of the epidermis to the dermis. Cyclin A2 (CCNA2) is involved in cell turnover. An increase in CCNA2 indicate improved cell turnover, which is reduced after UV challenge.

What is claimed is:

1. A personal care composition comprising:
a mineral UV filter;
a SPF enhancer comprising at least one of butyloctyl salicylate, ethylhexyl methoxycrylene, octyldodecyl neopentanoate, or Pongamia pinnata extract; and
an amino acid.

2. The personal care composition according to claim 1, wherein the mineral UV filter comprises a metal oxide.

3. The personal care composition according to claim 1, wherein the metal oxide is selected from zinc oxide, iron oxide, zirconium oxide, cerium oxide, and a combination of two or more thereof.

4. The personal care composition according to claim 1, wherein the SPF enhancer comprises butyloctyl salicylate and at least one of ethylhexyl methoxycrylene or Pongamia pinnata extract.

5. The personal care composition according to claim 1, wherein the amino acid comprises a plurality of amino acids selected from glycine, arginine, taurine, histidine, tyrosine, tryptophan, lysine, serine, citrulline, ornithine, creatine, diaminobutanoic acid, diaminoproprionic acid, alanine, aminobutyrate, asparagine, cysteine, cystine, glutamine, hydroxyproline, isoleucine, leucine, methionine, phenylalanine, proline, serine, taurine, threonine, tryptophan, tyrosine, valine, a salt thereof, and a combination of two or more thereof.

6. The personal care composition according to claim 5, wherein the plurality of amino acids is selected from glycine, arginine, taurine, histidine, tyrosine, tryptophan, a salt thereof, and a combination of two or more thereof.

7. The personal care composition according to claim 5, wherein the plurality of amino acids comprises glycine and/or a salt thereof, arginine and/or salt thereof, and taurine and/or a salt thereof.

8. The personal care composition according to claim 1, wherein the personal care composition further comprises a ceramide.

9. A personal care composition comprising:
a mineral UV filter;
a SPF enhancer comprising at least one of butyloctyl salicylate, ethylhexyl methoxycrylene, octyldodecyl neopentanoate, or Pongamia pinnata extract; and
a ceramide.

10. The personal care composition according to claim 9, wherein the mineral UV filter comprises a metal oxide.

11. The personal care composition according to claim 9, wherein the metal oxide is selected from zinc oxide, iron oxide, zirconium oxide, cerium oxide, and a combination of two or more thereof.

12. The personal care composition according to claim 9, wherein the personal care composition is substantially free or free of titanium oxide.

13. The personal care composition according to claim 9, wherein the personal care composition is substantially free or free of titanium compounds.

14. The personal care composition according to claim 9, wherein the SPF enhancer comprises butyloctyl salicylate and at least one of ethylhexyl methoxycrylene or Pongamia pinnata extract.

15. The personal care composition according to claim 9, wherein the ceramide comprises ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, ceramide NS dilaurate, or a combination of two or more thereof.

16. The personal care composition according to claim 9 further comprising an amino acid selected from glycine, arginine, taurine, histidine, tyrosine, tryptophan, lysine, serine, citrulline, ornithine, creatine, diaminobutanoic acid, diaminoproprionic acid, alanine, aminobutyrate, asparagine, cysteine, cystine, glutamine, hydroxyproline, isoleucine, leucine, methionine, phenylalanine, proline, serine, taurine, threonine, tryptophan, tyrosine, valine, a salt thereof, and a combination of two or more thereof.

17. The personal care composition according to claim 16, wherein the plurality of amino acids is selected from glycine, arginine, taurine, histidine, tyrosine, tryptophan, a salt thereof, and a combination of two or more thereof.

18. The personal care composition according to claim 16, wherein the plurality of amino acids comprises glycine and/or a salt thereof, arginine and/or salt thereof, and taurine and/or a salt thereof.

19. The personal care composition according to claim 18, wherein the personal care composition has a weight ratio of glycine and/or a salt thereof to arginine and/or a salt thereof to taurine and/or a salt thereof of about 1:24:45 to about 1:45:85.

20. A personal care composition comprising:
a mineral UV filter;
a SPF enhancer comprising at least one of butyloctyl salicylate, ethylhexyl methoxycrylene, octyldodecyl neopentanoate, or Pongamia pinnata extract;
an amino acid; and
a ceramide.
